(19) United States
(12) Patent Application Publication
(10) Pub. No.: US 2025/0383310 A1
(43) Pub. Date: Dec. 18, 2025

(54) WATER QUALITY INSPECTION METHOD AND FLOATING DEVICE OF WATER QUALITY INSPECTION BASED ON MICROCONTROLLER

(71) Applicant: Tiantuo FU, Beijing (CN)

(72) Inventor: Tiantuo FU, Beijing (CN)

(73) Assignee: Tiantuo FU, Beijing (CN)

(21) Appl. No.: 18/877,376

(22) PCT Filed: Apr. 26, 2023
(86) PCT No.: PCT/CN2023/090993

§ 371 Date: Dec. 20, 2024

(30) Foreign Application Priority Data
Jun. 21, 2022 (CN) 202210704983.1

(51) Int. Cl. G01N 27/04; G01N 27/07; G01N 33/18

(52) U.S. Cl. CPC G01N 27/046; G01N 27/07; G01N 33/1886

(57) ABSTRACT

Some embodiments of the disclosure provide a water quality inspection method and a floating device of water quality inspection based on a microcontroller. In some examples, the method includes: acquiring a first inspection value, adjusting the frequency and amplitude of an excitation voltage, acquiring a second inspection value, and characterizing the water quality condition at a second moment. The floating device includes an inspection module, an indication module, an analysis module, a power supply module, a control module, and a carrier. The inspection module has a first electrode and a second electrode. The power supply module, the analysis module, and the control module are integrated into the microcontroller. When the water quality inspection program is executed, the power supply module applies an excitation voltage between the first electrode and the second electrode, the analysis module analyzes the electrolyte content based on a current.

### CROSS REFERENCE TO RELATED APPLICATIONS

This application is the United State national stage entry under 37 U.S.C. 371 of PCT/CN2023/090993, filed on Apr. 26, 2023, which claims priority to Chinese application number 2022210704983.1, filed on Jun. 21, 2022, the disclosure of which are incorporated by reference herein in their entireties.

### FIELD OF THE DISCLOSURE

The disclosure relates generally to the field of water quality inspection technology. More specifically, the disclosure relates to water quality inspection methods and floating devices of water quality inspection based on microcontrollers.

### BACKGROUND

With the improvement of people's living standards, outdoor activities are becoming more and more popular, and more people are pursuing the quality of outdoor experiences. Some outdoor activities require the involvement of water areas, such as picnics or wild swimming, etc. Therefore, in order to enhance such outdoor experiences, people often need to quickly inspect the water quality. In addition, in some scientific research or engineering field sites where the requirements for inspection of the water quality are not high, but a quick inspection of the water quality is also needed.

In the prior art, the patent with application number CN202121013646.5 discloses a small unmanned boat for water quality inspection that may be used in various water environments to provide accurate real-time data information for water quality monitoring. The patent with application number CN202010114821.3 discloses a water quality monitoring device and a water quality inspection method that may remotely control the movement of the water quality monitoring device online to perform water quality inspection at multiple locations in the water, as well as retaining samples after inspection, or directly collecting water samples, the user does not need to doon-site inspection and sampling, the inspection result may be transmitted to user in real-time or stored in the water quality monitoring device, resulting in simplifying the process of water quality inspection process, saving the user's time, and significantly reducing work intensity. However, such devices in the prior art used for precise or real-time water quality inspection have complex structures, huge sizes, and high costs even if they have high inspection accuracy and of real-time monitoring capabilities, they are commonly used in large-scale scientific research or water quality monitoring projects, but not suitable for the needs of ordinary people for water quality inspection during outdoor activities.

In addition, the water quality is usually determined by acquiring the water's conductivity in the prior art, that is, by electrolyzing the water body. During electrolysis of the water body, metal ions such as calcium, magnesium, and iron in the water need to undergo electrochemical reactions on the surface of the electrolytic electrodes, easily leading to the formation and attachment of various metal reactants on the electrode surface, these metal reactants usually have weaker conductivity or even no conductivity compared to the electrode itself. Therefore, these metal reactants attached to the electrode surface will seriously affect the accuracy of water quality inspection if not removed. In the prior art, alternating current is commonly used as the electrolytic power source to reduce the formation of metal reactants and negative electrodes are added to adsorb the formed metal reactants. However, such approaches in the prior art usually place higher demands on the device, such as complex circuit designs, and such approaches in the prior art do not make specific adjustments to the electrolytic power source based on different water quality conditions in the water to adaptively adjust the voltage parameters according to the usage scenario of the electrode to reduce the formation of the aforementioned metal reactants.

Thus, it is particularly important to invent a device with simple structure and thereby suitable for the needs of rapid water quality inspection during people's outdoor activities, at the same time, it may adaptively adjust voltage parameters according to the usage scenario of the electrode to reduce the formation of the metal reactants and improve the inspection accuracy.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

The first aspect of the present disclosure is to provide a water quality inspection method, which is a method for inspecting water quality using a floating device of water quality inspection based on a microcontroller, the floating device includes a first electrode and a second electrode, the electrolyte content in the water is analyzed by the floating device via applying an alternating excitation voltage between the first electrode and the second electrode based on the intensity of the current formed between the first electrode and the second electrode, the inspection method includes: within a preset time interval, acquiring a first inspection value using the floating device for inspection, the first inspection value includes the electrolyte content acquired from the water by the floating device at a first moment; charactering the water quality condition at the first moment based on the first inspection value; adjusting the frequency and amplitude of the excitation voltage based on the first inspection value; acquiring a second inspection value by inspecting based on the adjusted excitation voltage, the second inspection value includes the electrolyte content acquired from the water by the floating device at a second moment; and characterizing the water quality condition at the second moment based on the second inspection value.

In the first aspect of the present disclosure, the conductivity of the water body may be inspected and the formation of reactants may be reduced by applying alternating excitation voltages. The water quality inspection value (i.e. the first inspection value) of the first moment is acquired within the preset time interval, adaptively adjusting the excitation voltage based on the first inspection value, the voltage parameters may be adaptively adjusted according to the usage scenario of the electrode to further reduce the formation of metal reactants. Then the water quality inspection value (i.e. the second inspection value) of a second moment is acquired by adaptively adjusting the excitation voltage, the impact of metal reactants on water quality inspection values may be reduced, thereby improving the inspection accuracy.

According to the water quality inspection method of the present disclosure, optionally, a frequency and an amplitude of the excitation voltage is adjusted based on an inspection model and the first inspection value. In this case, since the formation of metal reactants is related to the frequency and amplitude of the excitation voltage, the voltage parameters may be adaptively adjusted according to the usage scenario of the electrode to reduce the formation of reactants and to acquire relatively accurate water quality inspection values.

According to the water quality inspection method of the present disclosure, optionally, a frequency and an amplitude of the excitation voltage is acquired at the slowest formation of metal reactants on the electrode surface at a plurality of unit times after the first electrode and the second electrode are energized under a plurality of different electrolyte contents through machine learning and the inspection model is output. In this case, since machine learning has the advantages of identifying data trends and patterns that humans may miss, and processing various data formats in dynamic, high-capacity, and complex data environments, the correlation between excitation voltage and electrolyte content may be acquired, the manual intervention may be reduced and the convenience and accuracy of calculations may be improved through machine learning, thereby the frequency and amplitude of the excitation voltage corresponding to the slowest formation of metal reactants may be acquired (i.e., acquire the inspection model) by the floating device based on the first inspection value and the correlation, thus, the voltage parameters may be adaptively adjusted according to the usage scenario of the electrode and thereby reducing the formation of reactants and acquiring relatively accurate water quality inspection values.

According to the water quality inspection method of the present disclosure, optionally, the first moment and the second moment are within the preset time interval and the interval does not exceed the preset time interval. In this case, at least one first inspection value within a preset time interval may be acquired and adaptively adjust the voltage parameters according to the usage scenario of the electrode to acquire at least one second inspection value, thereby characterizing the water quality through the second inspection value and improving the accuracy of water quality inspection.

According to the water quality inspection method of the present disclosure, optionally, there are a plurality of the preset time intervals, and the plurality of preset time intervals are different from each other, a preset time interval is selected based on the first inspection value, and a plurality of the first inspection values and the second inspection values are acquired from the plurality of preset time intervals via the Sliding Window Method. In this case, the sliding time window method (i.e. Sliding Window Method) is used to acquire the first inspection value in the preset time intervals which are different from each other, thereby the frequency and amplitude of the excitation voltage are adjusted based on the first inspection value, and then the second inspection value is acquired by the adjusted excitation voltage, thus, the water quality inspection method may be repeatedly and cyclically executed, and accurate water quality inspection values may be obtained in real-time.

According to the water quality inspection method of the present disclosure, optionally, The first inspection value is an electrolyte content acquired by the floating device from a first water position at the first moment, the water quality condition is characterized at the first water position based on the first inspection value, the second inspection value is an electrolyte content acquired by the floating device from a second water position at the second moment, the water quality condition is characterized at the second water position based on the second inspection value. In this case, the water quality of a single designated water position may be acquired by inspecting the first position, the water quality of different positions in the water may be acquired by inspecting the first water position and the second water position.

According to the water quality inspection method of the present disclosure, optionally, the first inspection value further includes a temperature value acquired by the floating device from the water at the first moment, and the second inspection value further includes a temperature value acquired by the floating device from the water at the second moment. In this case, the excitation voltage may be adjusted based on the electrolyte content and the temperature value, thereby, the accuracy of the obtained water quality inspection value may be improved after the excitation voltage is adjusted.

For the above purpose, the second aspect of the present disclosure is to provide a floating device for water quality inspection based on a microcontroller, the microcontroller has an executable water quality inspection program, when the floating device executes the water quality inspection program, the water quality inspection is conducted using any of the water quality inspection methods described in the first aspect of the present disclosure, the floating device includes an inspection module, an indication module, an analysis module, a power supply module, a control module and a floatable carrier. The inspection module, the indication module, the analysis module, the power supply module, and the control module are installed on the carrier. The inspection module, the indication module, the analysis module, and the control module are electrically connected to each other and powered by the power supply module. The inspection module has a first electrode and a second electrode arranged opposite to each other, the inspection module is configured as followed: during water quality inspection, a first end of the first electrode and a first end of the second electrode are immersed in water, while a second end of the first electrode and a second end of the second electrode are extended and connected to the power supply module. The power supply module, the analysis module, and the control module are integrated into the microcontroller and, when the microcontroller executes the water quality inspection program they are operable as followed: the power supply module applies an excitation voltage between the second end of the first electrode and the second end of the second electrode, the analysis module analyzes the electrolyte content in the water based on the intensity of the current formed between the first electrode and the second electrode, the control module determines the water quality condition based on the electrode content and controls the indication module to emit an intensity indication based on the condition of the water quality.

In the second aspect according to the present disclosure, the water quality may be inspected via the inspection module, through the microcontroller integrated with the power supply module, the analysis module, and the control module, in this case, the floating device may be controlled during water quality inspection and the inspection data may be acquired, in addition, the water quality may be indicated in real-time and quickly through the indication module, thus, a device with a simple structure that is suitable for the needs of rapid water quality inspection during people's outdoor activities may be obtained.

According to the floating device of the present disclosure, optionally, when the microcontroller executes the water quality inspection program, the control module divides the water quality condition into a plurality of grades by setting preset values. In this case, the water quality may be divided by setting different preset values and based on different preset values, that is, different inspection accuracies may be set for water quality inspections, thereby facilitating the user to select different inspection accuracies for water quality inspections based on different water environments.

According to the floating device of the present disclosure, optionally, the indication module includes a plurality of indicators corresponding one-to-one with the grades, when the water quality is inspected, the control module controls the indicators corresponding to the grades to emit intensity indications based on the grades. In this case, the result of water quality inspection may be obtained through indicators, thereby facilitating the user to determine the condition of the water quality inspection via indicators corresponding one-to-one with different grades.

According to the floating device of the present disclosure, optionally, the indication module includes a rod-shaped support portion, and one end of the support portion is installed on a part of the carrier that floats on the water surface, and the indicator is mounted on the support portion. In this case, it is facilitated for the user to watch the indicator on the support portion to obtain the condition of the water quality inspection.

According to the floating device of the present disclosure, optionally, the microcontroller has a display and buttons, the display is used to display the preset values, the buttons are used to enter the preset values. In this case, the user may set the inspection accuracy of the floating device by typing in preset values via the buttons, and determine whether the input results are accurate or not by displaying the preset values on the display, thereby facilitating the user to select different inspection accuracies for water quality inspections based on different water environments, and improving the user's experiences.

According to the floating device of the present disclosure, optionally, the first electrode and the second electrode are in a strip shape, a first end of the first electrode is parallel to a first end of the second electrode, and the first electrode and/or the second electrode are made of any conductive materials in metal or graphite, a second end of the first electrode and a second end of the second electrode are wires. In this case, the first end of the first electrode is parallel to the first end of the second electrode, which facilitates the control module to calculate the conductivity coefficient between the first end of the first electrode and the first end of the second electrode, thus, the accuracy of the water quality inspection may be improved. In addition, the second end of the first electrode and the second end of the second electrode may connect the first end of the first electrode and the first end of the second electrode to the power supply module to obtain the excitation voltage, and may be wrapped by other mechanisms such as a pulley to immerse the first end of the first electrode and the second end of the second electrode in water at different depths.

According to the floating device of the present disclosure, optionally, the carrier is made of solid materials with a density less than that of water, and a part of the carrier that floats on the water surface has a chamber, the chamber is used to accommodate and secure the analysis module, the power supply module, and the control module. In this case, the floating device may remain floating on the water surface, thereby facilitating the user to obtain the result of water quality inspection by observing the indicator of the floating device during water quality inspection. In addition, the impact of the water body on the normal operation of the analysis module, the power supply module, and the control module of the floating device may be reduced during water quality inspection.

According to the floating device of the present disclosure, optionally, the device further includes a power module installed on the carrier, the power module is used to drive the floating device to move in the water. In this case, the floating device may be moved to different positions in the water for inspection by the power module, the floating device may also be moved to a position that is easy for withdrawal to facilitate its recovery.

According to the floating device of the present disclosure, optionally, the device further includes a pulley module installed on the carrier, the pulley module is used to wrap the second end of the first electrode and the second end of the second electrode so that the first end of the first electrode and the second end of the second electrode are immersed in the water at different depths. In this case, the first end of the first electrode and the first end of the second electrode of the inspection module may be placed at different depths in the water via the pulley module, thereby facilitating the user to perform water quality inspection on the water body at different depths to improve the accuracy of the inspection.

According to the floating device of the present disclosure, optionally, the device further includes a remote control, the remote control is used to remotely control the power supply module to drive the floating device to move in the water. In this case, the power module is controlled by the remote control, thereby facilitating the user to adjust and control the position of the floating device in the water.

According to the floating device of the present disclosure, optionally, the device further includes a remote control, the remote control is used to remotely control the pulley module to wrap the second end of the first electrode and the second end of the second electrode so that the first end of the first electrode and the second end of the second electrode are immersed in the water at different depths. In this case, the pulley module is controlled by the remote control, thereby facilitating the user to adjust and control the depth of immersion of the inspection module of the floating device in the water.

According to the present disclosure, a water quality inspection method and a portable floating device for water quality inspection based on a microcontroller may be provided, the floating device is simple in structure, thereby suitable for the needs of rapid inspection of water qualities during people's outdoor activities, at the same time, it's capable of adaptively adjusting the voltage parameters according to the usage scenario of the electrode to reduce the formation of reactants so as to improve the inspection accuracy.

### BRIEF DESCRIPTION OF THE DRAWINGS

Illustrative embodiments of the disclosure are described in detail below with reference to the attached drawing figures.

FIG. 1 is a flowchart showing the inspection method according to an example of the present disclosure.

FIG. 2a is a schematic diagram showing an embodiment 1 of the preset time interval in the inspection method according to an example of the present disclosure.

FIG. 2b is a schematic diagram showing an embodiment 2 of the preset time interval in the inspection method according to an example the present disclosure.

FIG. 2c is a schematic diagram showing an embodiment 3 of the preset time interval in the inspection method according to an example of the present disclosure.

FIG. 3 is a schematic diagram showing the application scenario of the floating device for water quality inspection based on the microcontroller according to an example of the present disclosure.

FIG. 4 is a schematic structural diagram showing the floating device for water quality inspection based on the microcontroller according to an example of the present disclosure.

FIG. 5 is a structural block diagram showing the floating device for water quality inspection based on the microcontroller according to an example of the present disclosure.

FIG. 6a is a schematic diagram showing the electrical connections between the power supply module, the indication module, the inspection module, the analysis module, and the control module in the floating device according to an example of the present disclosure.

FIG. 6b is a schematic diagram showing the electrical connections between the power supply module, the control module, the drive module, and the pulley module in the floating device according to an example of the present disclosure.

FIG. 7 is a schematic diagram showing the inspection principle of the inspection module in the floating device according to an example of the present disclosure.

FIG. 8 is a schematic structural diagram showing an embodiment of the inspection module in the floating device according to an example of the present disclosure.

FIG. 9 is a schematic structural diagram showing another embodiment of the inspection module in the floating device according to an example of the present disclosure.

FIG. 10 is a schematic structural diagram showing still another embodiment of the inspection module in the floating device according to an example of the present disclosure.

FIG. 11 is a schematic diagram showing the working scenario of the indication module in the floating device according to an example of the present disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

Hereinafter, preferred embodiments of the present disclosure will be described clearly and completely with reference to the accompanying drawings, obviously, the embodiments described are only a part of the embodiments of the present disclosure, not the entire embodiments. Based on the embodiments of the present disclosure, any other embodiments that are derived by an ordinary person skilled in the art without engaging in creative efforts shall also fall within the scope of protection of the present disclosure.

It should be noted that the terms “first”, “second”, “third”, and “fourth”, etc., used in the specifications, claims and accompanying drawings of the present disclosure are intended to distinguish different objects rather than to describe a specific order. In addition, the terms “include” and “have” and any variations thereof are intended to cover non-exclusive inclusions. For example, a process, method, system, product, or device that includes a series of steps or units is not limited to the listed steps or units, but may optionally include additional steps or units that are not listed, or may optionally include other steps or units inherent to the process, method, product, or device. In the following specifications, identical components are assigned the same symbols, and redundant explanations are omitted. In addition, the accompany drawings are merely schematic diagrams, and the proportions of the sizes or shapes of the components relative to each other may differ from actual implementations.

The first aspect of the present disclosure provides a water quality inspection method, which is a method for inspecting water quality using a floating device of water quality inspection based on a microcontroller. For the convenience of description hereinafter, it is sometimes referred to as an inspection method or simply a method etc. FIG. 1 is a flowchart showing the inspection method according to an example of the present disclosure.

In some examples, the floating device includes a first electrode and a second electrode, the electrolyte content in the water is analyzed by the floating device by applying an alternating excitation voltage between the first electrode and the second electrode and based on the intensity of the current formed between the first electrode and the second electrode. In this case, the conductivity of the water body may be detected and the formation of reactants may be reduced by applying alternating excitation voltages, specifically, the floating device of the present disclosure may be referred to contents described in the second aspect of the present disclosure.

As shown in FIG. 1, the water quality inspection method of the present disclosure may include: obtaining a first inspection value at a first moment (step S100); characterizing the water quality situation at the first moment based on the first inspection value (step S200); adjusting the excitation voltage based on the first inspection value (step S300); acquiring a second inspection value based on the adjusted excitation voltage (step S400); and characterizing the water quality at a second moment based on the second inspection value (Step S500).

In some examples, steps S100 to S400 may be performed within a preset time interval. In other examples, step S500 may also be performed within the preset time interval.

In some examples, the floating device may be used to acquire the first inspection value and the second inspection value.

In some examples, the first inspection value may include the electrolyte content acquired by the floating device from the water at the first moment.

In some examples, “characterization” may also be understood to meaning of representation or indication. The specific forms or ways of characterization may include but not limited to numerical values, light color indications, light intensity indications, sound indications, or image indications, etc.

In some examples, step S200 may not be obligatory. For example, when the floating device is used for the first time (i.e., in case that the floating device has not been used before), deposits such as metal reactants etc. have not been formed on the surface of the electrode has not yet formed, therefore, the first inspection value may be used to characterize the water quality. When the floating device is used not for the first time (i.e., in case that the floating device has been used before), due to deposits such as the metal reactants etc. may have been formed on the surface of the electrode, it is less accurate to use the first inspection value to characterize the water quality, that is, in the present disclosure, the second inspection value may be used only to characterize water quality.

In some examples, the alternating excitation voltage may refer to a voltage that varies periodically with time in terms of both magnitude and direction.

In some examples, step S300 may specifically involves adjusting the frequency and amplitude of the excitation voltage based on the first inspection value.

In some examples, the second inspection value includes the electrolyte content acquired by the floating device from the water at the second moment.

In the inspection methods according to the present disclosure, the water quality inspection value (i.e., the first inspection value) of the first moment is acquired within the preset time interval, and the excitation voltage is adaptively adjusted based on the first inspection value, the voltage parameters may be adaptively adjusted according to the usage scenario of the electrode to reduce the formation of metal reactants. The second water quality inspection value at the second moment (i.e., the second inspection value) is acquired after the excitation voltage is adaptively adjusted, after the voltage parameters are adaptively adjusted according to the usage scenario of the electrode to reduce the formation of metal reactants, subsequent inspection may be done to obtain relatively accurate water quality inspection values, thereby the inspection accuracy may be improved.

In addition, in step S300, the excitation voltage required to be adjusted may be the preferred excitation voltage corresponding to the slowest metal reactants formed on the electrode surface after the first electrode and the second electrode are energized, including voltage frequency and amplitude. In this case, the excitation voltage may be adjusted in real-time to minimize the formation of metal reactants on the electrode surface, thereby enabling the floating device to obtain relatively accurate water quality inspection values. In other words, the preferred excitation voltage may be pre-set in the water quality inspection program of the floating device, it is invoked when adjustments are made based on the first inspection value, and the corresponding optimal excitation voltage may be selected according to the different first inspection values, therefore, the second inspection value may be acquired by adjusting the excitation voltage to characterize the water quality situation, that is, the accuracy of inspection may be improved.

In addition, in some examples, the excitation voltage that needs to be adjusted may be acquired through the ways of machine learning. Specifically, the frequency and amplitude of the excitation voltage are acquired at the slowest formation of metal reactants on the electrode surface at a plurality of unit times after the first electrode and the second electrode are energized under a plurality of different electrolyte contents through machine learning and the inspection model is output. In this case, since machine learning has the advantages of identifying data trends and patterns that humans may miss, and processing various data formats in dynamic, high-capacity, and complex data environments, the correlation between excitation voltage and electrolyte content may be acquired, then the frequency and amplitude of the excitation voltage corresponding to the slowest formation of metal reactants may be acquired (i.e., acquire the inspection model) based on the correlation, thus, the voltage parameters may be adaptively adjusted according to the usage scenario of the electrode and thereby reducing the formation of reactants and acquiring relatively accurate water quality inspection values.

In some examples, acquiring the excitation voltage that needs to be adjusted through machine learning may be done in a laboratory. Specifically, firstly, the first electrode and the second electrode may be placed in liquids with different electrolyte contents respectively, then excitation voltages are applied, the formation of metal reactants on the electrode surface is recorded, the excitation voltage is adjusted, n times are repeated, for example, based on common electrolyte components (such as at least one electrolyte among iron ions, magnesium ions, sodium ions, calcium ions, or potassium ions is contained) may be repeated at least 120 times (i.e., related to the arrangement and combination of ion types and concentration levels, the more repetitions, the higher the accuracy), the recorded data is input into the machine learning inspection model and the inspection model is trained.

In some examples, the liquids with different electrolyte contents may refer to different types of electrolytes. In other examples, the liquids with different electrolyte contents may refer to different electrolyte contents. In other examples, the liquids with different electrolyte contents may refer to different types and contents of electrolytes.

In some examples, the machine learning methods may include but not be limited to at least one of the Linear Regression Algorithm, the Support Vector Machine Algorithm, the Nearest Neighbor/k-nearest Neighbor Algorithm, the Logistic Regression Algorithm, the Decision Tree Algorithm, the k-means Algorithm, the Random Forest Algorithm, the Naive Bayesian Algorithm, the Dimensionality Reduction Algorithm or the Gradient Enhancement Algorithm.

In some examples, the inspection model may be at least one of the coefficient, function, curve, or image that reflects the correlation between the excitation voltage, the inspection value, and the metal reactant. In some examples, the inspection model may be preset in the form of a program in the floating device.

In some examples, the frequency and amplitude of the excitation voltage may be adjusted based on the inspection model and the first inspection value. In this case, the voltage parameters may be adaptively adjusted according to the usage scenario of the electrode to reduce the formation of reactants and acquire relatively accurate water quality inspection values.

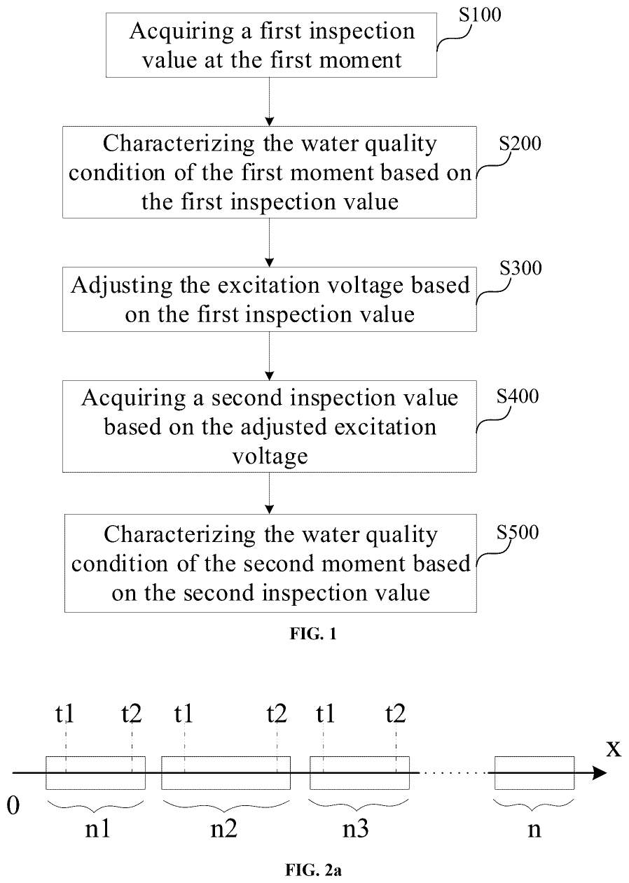

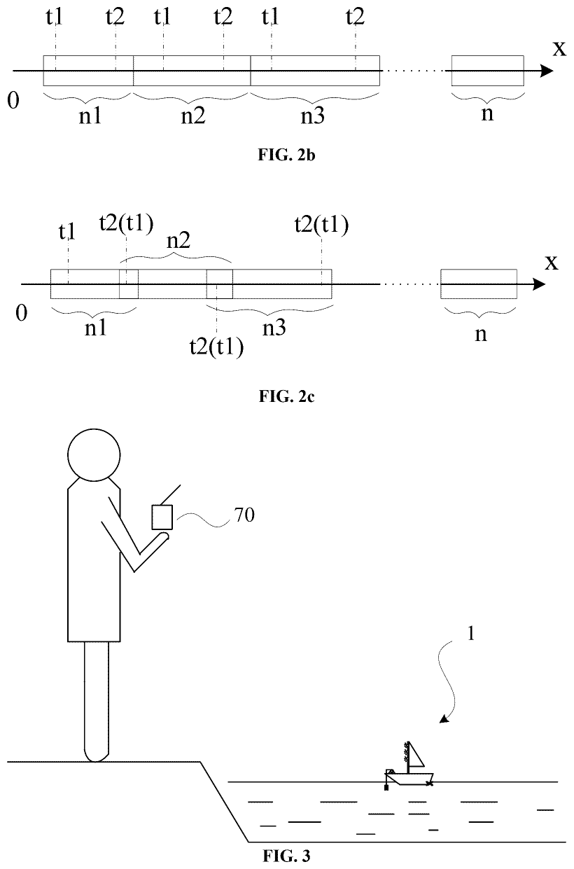

FIG. 2a is a schematic diagram showing embodiment 1 of the preset time interval in the inspection method according to an example of the present disclosure. FIG. 2b is a schematic diagram showing embodiment 2 of the preset time interval in the inspection method according to an example of the present disclosure. FIG. 2c is a schematic diagram showing embodiment 3 of the preset time interval in the inspection method according to an example of the present disclosure.

As shown in FIG. 2a, FIG. 2b, or FIG. 2c, in some examples, the first moment and the second moment may be within a preset time interval and the interval does not exceed the preset time interval. For example, a plurality of t1 (i.e., the first moment) and t2 (i.e., the second moment) may be located within the preset time interval n1, n2, n3 . . . or n respectively, and the interval between the two does not exceed any preset time interval. In this case, at least one first inspection value may be acquired within the preset time interval and the voltage parameters are adaptively adjusted according to the usage scenario of electrodes to acquire at least one second inspection value, thereby characterizing the water quality situation via the second inspection value to improve the accuracy of water quality inspection.

In some examples, the preset time interval may be plural, and a plurality of preset time intervals may be different from each other, for example, the preset time interval n1 may be 1 second, the preset time interval n2 may be 5 seconds, and the preset time interval n3 may be 10 seconds.

As shown in FIG. 2a, in some examples, the two adjacent preset time intervals may be independent of each other, i.e., not continuous or not overlapping. In this case, the floating device may be used to conduct water quality inspection at different independent time periods, and relatively accurate water quality inspection values may be acquired.

As shown in FIG. 2b, in other examples, the two adjacent preset time intervals may be continuous.

As shown in FIG. 2c, in other examples, the two adjacent preset time intervals may be continuous and may have overlapping regions. In this case, the first moment in one preset time interval may be used as the second moment of the previous preset time interval, or the second moment of the previous preset time interval may be used as the first moment of the next preset time interval, and a plurality of water quality inspection values are obtained using the Sliding Window Method, thus, continuous water quality inspection may be performed. For example, t1 (i.e., the first moment) of the preset time interval n2 may be used as t2 (i.e., the second moment) of the preset time interval n1, and t2 (i.e., the second moment) of the preset time interval n1 may be used as t1 (i.e., the first moment) of the preset time interval n2.

In some examples, a plurality of water quality inspection values may be obtained using the Sliding Window Method in each embodiment as shown in FIG. 2a, FIG. 2b, and FIG. 2c. In this case, appropriate preset time intervals may be selected for continuous water quality inspection based on different inspection models, thereby, the continuous water qualities may be obtained.

In some examples, the preset time interval may be designed based on the inspection model, that is, the selection of the preset time intervals may be made according to the inspection model. For example, when there is a smaller amount of metal reactants formed on the electrode surface of the floating device, a plurality of preset time intervals with an interval less than 5 seconds may be selected, when there is a larger amount of metal reactants formed on the electrode surface of the floating device, a plurality of preset time intervals with an interval greater than 5 seconds may be selected.

In some examples, the preset time interval may be selected based on the first inspection value, and a plurality of first inspection values and second inspection values may be obtained from a plurality of preset time intervals through the Sliding Window Method. In this case, the sliding time window method (i.e., Sliding Window Method) is used to obtain the first inspection value from the plurality of preset time intervals which are different from each other, and thereby the frequency and amplitude of the excitation voltage are adjusted based on the first inspection value, and then the second inspection value is acquired based on the adjusted excitation voltage, thus, the water quality inspection method may be repeatedly and cyclically performed, and accurate water quality inspection values may be obtained in real-time.

In some examples, the first inspection value may also represent an electrolyte content acquired by the floating device from a first water position at a first moment, and the water quality of the first water position may be characterized based on the first inspection value. In this case, the water quality condition of a single designated water area may be obtained.

In some examples, the first inspection value may also include a temperature value acquired by the floating device from the water at the first moment. In this case, the excitation voltage may be adjusted based on the electrolyte content and the temperature value, thereby, the accuracy of the obtained water quality inspection values may be improved after the excitation voltage is adjusted.

In some examples, the second inspection value may also represent an electrolyte content acquired by the floating device from a second water position at a second moment, and the water quality condition at the second water position may be characterized based on the second inspection value. In this case, by detecting the positions of the first and second water areas, the water quality conditions at different positions of the water may be obtained.

In some examples, the second inspection value may also include a temperature value acquired by the floating device from the water at the second moment. In this case, the water quality condition may be obtained accurately.

The second aspect of the present disclosure relates to a floating device for water quality inspection based on a microcontroller, sometimes may referred to as a floating device for short hereunder. In some examples, the floating device may have an executable water quality inspection program. In some examples, the floating device may perform water quality inspection using any of the inspection methods described in the first aspect of the present disclosure when the water quality inspection program is executed.

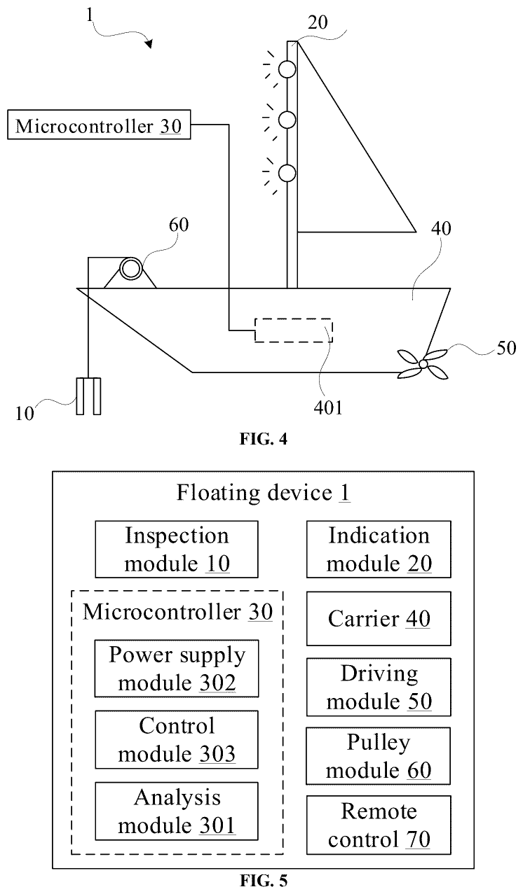

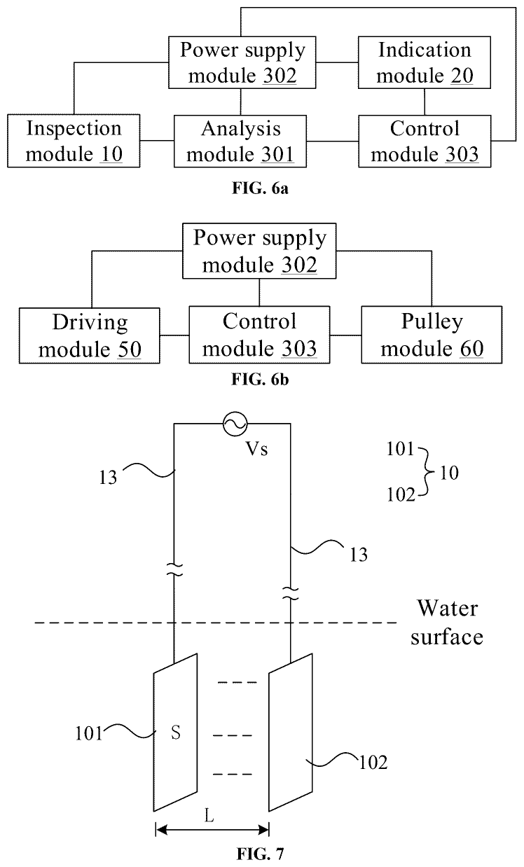

FIG. 3 is a schematic diagram of the application scenario showing a floating device 1 for the water quality inspection based on the microcontroller according to an example of the present disclosure. FIG. 4 is a schematic structural diagram showing the floating device 1 for the water quality inspection based on the microcontroller according to an example of the present disclosure. FIG. 5 is a structural diagram showing the floating device 1 for the water quality inspection based on a microcontroller 30 according to an example of the present disclosure. FIG. 6a is a schematic diagram showing the electrical connections between a power supply module 302, an indication module 20, an inspection module 10, an analysis module 301, and a control module 303 in the floating device 1 according to an example of the present disclosure. FIG. 6b is a schematic diagram showing the electrical connections between the power supply module 302, the control module 303, a driving module 50, and a pulley module 60 in the floating device 1 according to an example of the present disclosure.

As shown in FIG. 4 and FIG. 5, the floating device 1 may be a portable floating device 1 for the water quality inspection based on the microcontroller 30. The floating device 1 may include an inspection module 10, an indication module 20, a microcontroller 30, and a floatable carrier 40.

In some examples, the microcontroller 30 may include an analysis module 301, a power supply module 302, and a control module 303. In other words, in some examples, the power supply module 302, the analysis module 301, and the control module 303 may be integrated into the microcontroller 30 (to be described in detail later).

In some examples, the inspection module 10, the indication module 20, the analysis module 301, the power supply module 302, and the control module 303 may be installed on the carrier 40.

As shown in FIG. 6a, in some examples, the inspection module 10, the indication module 20, the analysis module 301, and the control module 303 may be electrically connected to each other. In some examples, the power supply module 302 may supply power to the inspection module 10, the indication module 20, the analysis module 301, and the control module 303.

In some examples, the inspection module 10 (to be described in detail later) may be used to inspect the electrolyte content in the water, the inspection principle of the inspection module 10 may be as follows: firstly, applying excitation voltage to the inspection module 10, a conductive circuit is formed while the inspection module 10 is in contact with the water body, and then obtaining the magnitude of the current formed between the inspection module 10 and the water body to determine the conductive capacity (i.e., the conductivity, indicating the electrolyte content) of water quality, finally, determining the water quality condition based on the conductive capacity of the water quality, i.e., determining the water quality based on the electrolyte content, under common circumstances, the higher the electrolyte content in the water is, the worse the water quality condition is.

In some examples, the indication module 20 (to be described in detail later) may be used to indicate the water quality condition, for example, through sound, light intensity, or indicator light color, etc.

In some examples, the analysis module 301 may be used to obtain the magnitude of the current formed between the inspection module 10 and the water body. In some examples, the analysis module 301 may input the information of the obtained current magnitude into the control module 303.

In some examples, the control module 303 may be used to control the operation of the indication module 20 based on the information of current magnitude of the inspection module 10 obtained by the analysis module 301.

In some examples, the power supply module 302 may be used to supply power to the indication module 20, the inspection module 10, the analysis module 301, and the control module 303. For example, the power supply module 302 may supply power to the inspection module 10 to obtain the required excitation voltage for inspecting the water quality.

In some examples, the floatable carrier 40 (to be described in detail later) enables the floating device 1 to float on the water surface. In some examples, the carrier 40 may be used to carry and install electronic components such as the inspection module 10, indication module 20, analysis module 301, power supply module 302, and the control module 303 etc., enabling the floating device 1 to float on the water surface for conducting the water quality inspection.

As shown in FIG. 4 and FIG. 5, in some examples, the floating device 1 may further include a driving module 50 (to be described in detail later), a pulley module 60 (to be described in detail later), and a remote control 70 (to be described in detail later). As shown in FIG. 6b, in some examples, the power supply module 302, the control module 303, the driving module 50, and the pulley module 60 may be electrically connected to each other. In this case, the control module 303 may control the driving module 50 and the pulley module 60, and also the power supply module 302 may supply power to the control module 303, the driving module 50, and the pulley module 60, thereby facilitating the user to conduct water quality inspection in different water bodies and depths by the floating device 1.

In the present disclosure, the water body may be inspected (i.e., water quality inspection) by the inspection module 10, through the microcontroller 30 integrated with the power supply module 302, analysis module 301, and the control module 303, the floating device 1 may be controlled and the inspection data may be obtained during water quality inspection. In addition, the water quality may be indicated in real-time and quickly via the indication module 20. In addition, through the driving module 50, the pulley module 60, and the remote control 70, it is facilitated for the user to control the floating device 1 to conduct water quality inspection at different water positions and depths. Thus, a device with a simple structure that is suitable for the needs of quickly inspection of water quality during people's outdoor activities may be obtained.

The various components of the floating device 1 will be described in detail hereunder.

As shown in FIG. 4 and FIG. 5, in some examples, the floating device 1 may include a microcontroller 30.

As shown in FIG. 5, in some examples, the power supply module 302, the analysis module 301, and the control module 303 may be integrated on the microcontroller 30. The microcontroller 30 according to the present disclosure is also known as a Microcontroller Unit (MCU), The microcontroller may be classified from different aspects: According to the width of the data bus, it may be classified into three types: 8-bit, 16-bit, and 32-bit. According to the storage structure, it may be classified into Harvard structure and Von Neumann structure. According to the category of embedded program memory, it may be classified into OTP, Mask, EPROM/EEPROM, and Flash memory. According to the instruction structure, it may be further classified into CISC (Complex Instruction Set Computer) and RISC (Reduced Instruction Set Computer).

In some examples, the microcontroller 30 according to the present disclosure may adopt microcontrollers 30 including but not limited to PIC series, ARM series, 8051 series, AVR series, and MSP series. For example, the microcontroller 30 according to the present disclosure may include the following microcontrollers: STM32F103x, ATmega328, PIC16F877A, Attiny85, MSP430, ESP8266, ESP32, ATMEGA32U4, STM8, LPC1768, and etc.

In some examples, the analysis module 301 may be at least one of the data input port, analog-to-digital/digital-to-analog conversion circuit, or the signal amplification circuit of the microcontroller 30. In some examples, the analysis module 301 may be electrically connected to the inspection module 10. In this case, the current signals or data of the inspection module 10 may be obtained by the analysis module 301.

In some examples, the control module 303 may be a control chip or a logic arithmetic unit of the microcontroller 30. In this case, the control module 303 may be used to control the operation of the indication module 20 based on the information of the current magnitude of the inspection module 10 obtained from the analysis module 301.

In some examples, the microcontroller 30 according to the present disclosure may have an executable water quality inspection program. In some examples, the water quality inspection program may be pre-edited and embedded into the microcontroller 30, or it may be edited in real-time on-site. In this case, the pre-edited water quality inspection program may be batch packaged and embedded into the storage medium of the microcontroller 30, thereby facilitating the mass production of portable floating device 1 of the water quality inspection with consistent functions. In addition, through the on-site real-time editing of the water quality inspection program, it may facilitate the user to program the floating device 1 according to their needs to meet different situations of the water quality inspection, such as setting inspection time, accuracy, etc.

In some examples, when the microcontroller 30 executes the water quality inspection program, the control module 303 may divide the water quality into a plurality of grades by setting preset values. In this case, the water quality is divided by setting different preset values and based on different preset values, i.e., different inspection accuracies are set for water quality inspections, thereby facilitating the user to select different inspection accuracies for water quality inspections based on different water environments. For example, the TDS (Total dissolved solids) value of natural water is generally between 30 ppm and 300 ppm, while the TDS value of wastewater is usually above 300 ppm. For example, the preset values may be set to any one or a plurality of 30 ppm, 60 ppm, 100 ppm, 150 ppm, 200 ppm, 250 ppm, 300 ppm, etc. For example, when the preset values are 150 ppm, 0 ppm to 60 ppm may be set as the first grade, indicating good water quality. 60 ppm to 120 ppm may be set as the second grade, indicating average water quality. 150 ppm or above may be set as the third level, indicating poor water quality. For example, when the preset values are 300 ppm, 0 ppm to 200 ppm may be set as the first grade, indicating good water quality. 200 ppm to 300 ppm may be set as the second grade, indicating average water quality. 300 ppm or above may be set as the third grade, indicating poor water quality.

In some examples, when the microcontroller 30 executes the water quality inspection program, the floating device 1 may perform water quality inspection using any of the inspection methods in the first aspect of the present disclosure.

In some examples, the microcontroller 30 may have a display and buttons (not shown), the display may be used to display the preset value, and the buttons may be used to enter the preset values. In this case, the inspection accuracy of the floating device 1 may be set by a user by entering the preset values through the buttons, and determine whether the input result is accurate or not through the preset values displayed on the display, thereby facilitating the user to choose different inspection accuracies according to different water environments for water quality inspection, and improving the user experience. In some examples, the display may also be used to display the water quality condition, for example, a pre-set water quality condition may be represented by a number or a letter, while display of the number or the letter represents the corresponding water qualities.

In some examples, the power supply module 302 may include power sources and power control circuits. In some examples, the power supply may be detachably mounted in the microcontroller 30. In this case, the power supply may be used to supply electrical energy, and the power control circuits may control the power sources to provide corresponding voltages or currents based on different electronic components, for example, an alternating voltage for water quality inspection may be supplied to the inspection module 10, or a stable direct current working voltage may be supplied to the control module 303.

In some examples, the analysis module 301, the power supply module 302, and the display of the microcontroller 30 may be controlled by the control module 303. In this case, the control module 303 may be programmed to control the working status of the analysis module 301, the power supply module 302, and the display

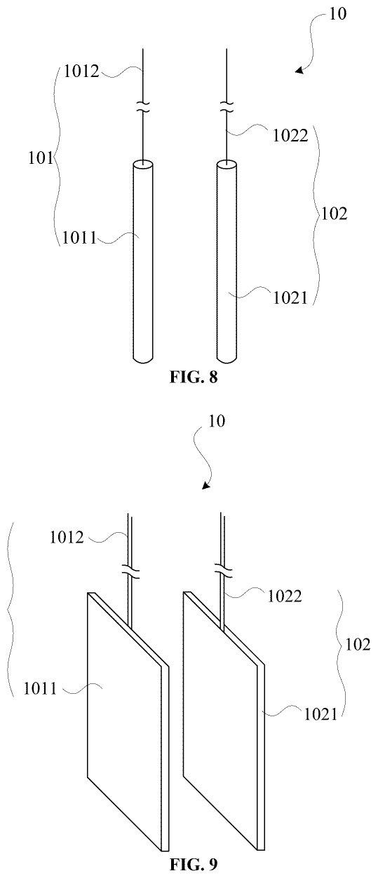

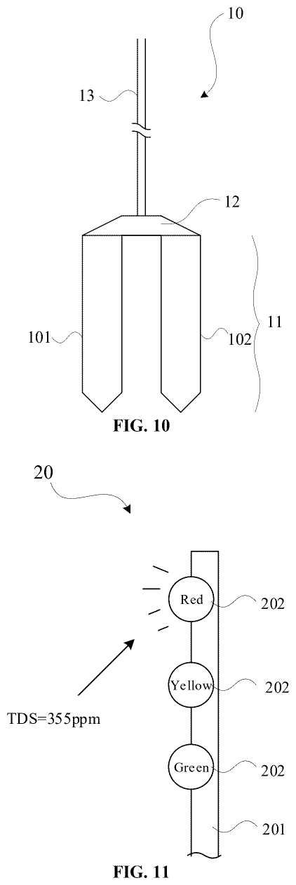

FIG. 7 is a schematic diagram showing the inspection principle of the inspection module 10 in the floating device 1 according to an example of the present disclosure. FIG. 8 is a schematic structural diagram showing an embodiment of the inspection module 10 in the floating device 1 according to one example of the present disclosure. FIG. 9 is a schematic structural diagram showing another embodiment of the inspection module 10 in the floating device 1 according to an example of the present disclosure. FIG. 10 is a schematic structural diagram showing still another embodiment of the inspection module 10 in the floating device 1 according to an example of the present disclosure.

As mentioned above, the floating device 1 may include an inspection module 10.

As shown in FIG. 7, FIG. 8, or FIG. 9, in some examples, the inspection module 10 may have a first electrode 101 and a second electrode 102 arranged opposite to each other. In some examples, the first electrode 101 and the second electrode 102 are in a strip shape. In some examples, the first electrode 101 may include a first end 1011 and a second end 1012. In some examples, the second electrode 102 may include a first end 1021 and a second end 1022.

As shown in FIG. 8 or FIG. 9, in some examples, when the water quality is inspected, the inspection module 10 may be configured as follows: the first end 1011 of the first electrode 101 and the first end 1021 of the second electrode 102 are immersed in water, the second end 1012 of the first electrode 101 and the second end 1022 of the second electrode 102 are extended and connected to the power supply module 302. In this case, the inspection module 10 may be in contacted with the water body to form a circuit, and a current formed between the inspection module 10 and the water body may be collected by applying excitation voltages on the first electrode 101 and the second electrode 102, and then the magnitude of the formed current via the analysis module 301 is to be analyzed, thereby the result of water quality inspection may be obtained.

In some examples, the first end 1011 of the first electrode 101 may be substantially parallel to the first end 1021 of the second electrode 102. In this case, the parallelism between the first end 1011 of the first electrode 101 and the first end 1021 of the second electrode 102 may facilitate the control module 303 to calculate the conductivity coefficient between the first end 1011 of the first electrode 101 and the first end 1021 of the second electrode 102, thereby the accuracy of water quality inspection may be improved.

As shown in FIG. 8 or FIG. 9, in some examples, the first end 1011 of the first electrode 101 and the first end 1021 of the second electrode 102 may be substantially parallel at least one of two conductors of at least one shape of cylinders, rectangles or sheet-like.

In some examples, the first end 1011 of the first electrode 101 and the first end 1021 of the second electrode 102 may be made of a conductive material in metal or graphite. In some examples, the second end 1012 of the first electrode 101 and the second end 1022 of the second electrode 102 may be conductive wires, such as flexible conductive wires. In some examples, the second end 1012 of the first electrode 101 and the second end 1022 of the second electrode 102 may be made of conductive materials with lower electrical resistivity. In this case, the first ends of the two electrodes are immersed in water, which may be used to chemically react with the electrolyte in water after excitation voltage is applied to form an electric current. In addition, the second ends of the two electrodes may be used to extend and connect to the power supply module 302, and also be electrically connected with the analysis module 301. Thus, an excitation power may be applied to the first end of the two electrodes via the second end of the two electrodes, and the current formed between the inspection module 10 and the water body after applying the excitation power may be obtained via the analysis module 301. In addition, the second end 1012 of the first electrode 101 and the second end 1022 of the second electrode 102 may connect the first end 1011 of the first electrode 101 and the first end 1021 of the second electrode 102 to the power supply module 302 to obtain an excitation voltage, and also may be wrapped by other mechanisms such as a pulley to immerse the first end 1011 of the first electrode 101 and the first end 1021 of the second electrode 102 in water at different depths. In addition, when the resistivities of the second ends of the two electrodes are lower, the conductivities are stronger, thereby the accuracy of detecting electrolytes in the water body may be improved.

In some examples, the second end 1012 of the first electrode 101 and the second end 1022 of the second electrode 102 may be wrapped around the pulley module 60. In some examples, the first end 1011 of the first electrode 101 of the inspection module 10 and the first end 1021 of the second electrode 102 may be placed in the water at different depths by controlling the pulley module 60. Thereby it is facilitated for the user to inspect the water body at different depths to improve the accuracy of inspection.

As shown in FIG. 10, in some examples, the first end 1011 of the first electrode 101 and the first end 1021 of the second electrode 102 may have a parallel portion 11 and a non-parallel portion 12. In some examples, the parallel portions 11 of the two electrodes may be contacted with the water body directly, that is, exposed in the water body. In some examples, the non-parallel portions 12 of the two electrodes may be gradually convergent and be connected to the second end of the two electrodes for electricity, and the outer part of the non-parallel portion 12 may be wrapped with insulation materials, that is, not in contact with the water body. In some examples, the outer parts of the second end 1012 of the first electrode 101 and the second end 1022 of the second electrode 102 are both wrapped with insulation materials and bundled in an insulated flexible sleeve 13. In this case, cable settings may be reduced and it is facilitated for the user to use the floating device 1 for water quality inspection.

In some examples, the excitation voltages applied to the two electrodes of the inspection module 10 may be either a DC voltage or an AC voltage. Preferably, the voltages applied to the two electrodes of the inspection module 10 may be the AC voltages (alternating voltage). In this case, the alternating excitation voltage may enable each electrode to repeatedly undergo oxidations or reduction reactions when the two electrodes are in contact and reacting with the water body, thereby the polarization issue that may arise in the two electrodes of the inspection module 10 may be reduced.

In some examples, the conductivity of the water body is determined by the inspection module 10 via an electrode method. The specific calculation principle of determining the conductivity of the water body using the electrode method may be as follows:
According to Ohm's law, when the temperature is constant,

$$R = ρ L / S$$

Here, R represents a resistance of the water body between the two electrodes of the inspection module 10, ρ represents a resistivity, L represents a distance between the two electrodes (See FIG. 7), S represents a cross-sectional area of the parallel portion 11 of the two electrodes (See FIG. 7). Since S and L are constant and unchanging, L/S represents a constant, known as an electrical conductance constant Q, i.e.,

$$R = ρ Q$$

An electrical conductance T is inversely proportional to the resistance R, i.e.,

$$T = 1 / R$$

An electrical conductivity K is inversely proportional to the resistivity p, i.e.,

$$K = 1 / ρ$$

It may be obtained from the above formulas: K=1/ρ=Q/R

Thus, in some examples, the excitation voltage is applied to the first end of the two electrodes of the inspection module 10 that are immersed in the water, and a tiny current formed between the two electrodes of the inspection module 10 and the water body is obtained through the analyzing module 301. In this case, the electrical conductivity of the water body may be obtained through the electrode method, thereby the electrolyte content in the water may be obtained so as to determine the water quality conditions.

In some examples, prior to the use of the electrode method, the two electrodes of the inspection module 10 may be calibrated with a standard solution to obtain the electrical conductance constant Q of the two electrodes of the inspection module 10, then the water quality inspection is performed to obtain the resistance R of the water body, finally, the electrical conductivity K of the water body may be obtained.

In the present embodiment, the content of water electrolytes may refer to the total dissolved solids (TDS) in the water with a measurement unit being milligrams per liter (mg/L), which indicates how many milligrams of dissolved solids are present in 1 liter of water. TDS may be used to represent the measurement of all solid substances dissolved in the water, including minerals, salts, and tiny metallic substances dissolved in the water. In some examples, the measurement unit of the TDS may be ppm (parts per million concentration). In some examples, the salt content in a solution may be inferred by the electrical conductivity, the purer the water, the less soluble solids, the higher the resistance, the lower the conductance capacity (or electrical conductivity), therefore, pure water has almost no conductivity. In some examples, there is a direct relationship between TDS value and the conductivity, the TDS value generally is 0.55 times of the conductivity, i.e. TDS=0.55K. In this case, the TDS value of the water body may be obtained via the inspection module 10 and the analyzing module 301, thus, the water quality may be obtained.

In some examples, the TDS value of the water body may be obtained via the inspection module 10 and the analysis module 301, and the TDS value may be transformed to data by the analysis module 301 and transmitted to the control module 303. In this case, the control module 303 may control the indication module 20 to issue corresponding intensity indications based on the TDS value, i.e., the water quality, thereby facilitating the user to determine the water quality so as to improve the experience during outdoor activities relates to water.

As shown in FIG. 4 and FIG. 5, in some examples, the floating device 1 may include an indication module 20. FIG. 11 is a schematic diagram of the working scenario showing the indication module 20 in the floating device 1 according to an example of the present disclosure.

As shown in FIG. 11, in some examples, the indication module 20 may include a rod-shaped support portion 201, and one end of the support portion 201 may be installed on the floating part of the carrier 40 on the water surface, and an indicator 202 may be mounted on the support portion 201. In some examples, the indicator 202 may be a colored luminous device. In this case, it may facilitate the user to observe the indicator 202 on the support member 201 to obtain the situation of the water quality inspection.

In some examples, the indicator 202 may also be any other device with a feature discrimination. For example, the indicator 202 may be a sound producing device with a sound discrimination.

As shown in FIG. 11, in some examples, the indication module 20 may have a plurality of indicators 202, and the plurality of indicators 202 may correspond one-to-one to the grades of the aforementioned water quality, when water quality is inspected, the control module 303 emits intensity indications based on the grade control and the indicator 202 corresponding to the grades. For example, the indicators 202 may use three kinds of lighting devices in red, yellow, and green to correspond to three conditions of good, average, and poor water quality respectively. In this case, the result of water quality inspection may be obtained by the indicators 202, thereby facilitating the user to determine the condition of the water quality inspection via the indicator 202 corresponding to different grades one-to-one.

In some examples, the indicator 202 may not be installed on the carrier 40, for example, the indicator 202 may be integrated with a remote control 70 and communicate wirelessly with the control module 30. In this case, it may reduce the inconvenience for the user to watch the indicator 202 while conducting water quality inspection in open water areas, thereby improving the convenience.

As shown in FIG. 4 and FIG. 5, in some examples, the floating device 1 may include a carrier 40.

In some examples, the carrier 40 may be made of solid materials with a density less than water, such as foamed cottons or light plastics. In this case, the floating device 1 may remain floating on the water surface, thereby facilitating the user to obtain the result of water quality inspection by observing the indicator 202 of the floating device 1 when conducting water quality inspection in open water areas.

In some examples, the carrier 40 may be used to mount the inspection module 10, the indication module 20, the analysis module 301, the power supply module 302, and the control module 303. In some examples, the carrier 40 may be further used to mount other components of the floating device 1, such as the driving module 50, the pulley module 60, etc. In this case, the floating device 1 may float on the water surface and protect each component of the floating device 1 from the influence of the water body.

As shown in FIG. 4, in some examples, the part of the carrier 40 that floats on the water surface may have a chamber 401, which may be used to accommodate and secure the microcontroller 30 (including the analysis module 301, the power supply module 302, and the control module 303). In some examples, the chamber 401 may be a closed space. In some examples, the chamber 401 of the carrier 40 may be sealed after accommodating and securing each electronic components of the floating device 1, such as the analysis module 301, the power supply module 302, and the control module 303. In this case, through this chamber 401, the impact of the water body on the normal operation of the analysis module 301, the power supply module 302, and the control module 303 of the floating device 1 may be reduced during water quality inspection.

As shown in FIG. 4, in some examples, the carrier 40 may be shaped like a boat. In this case, it may provide aesthetic pleasure to the user, in addition, the boat-shaped body may cooperate with the power supply module 50 to move even more smoothly in the water.

In some examples, the shape of carrier 40 may be arbitrary. In this case, the shape of the carrier 40 may be designed according to each component of the floating device 1, thereby facilitating the floating device 1 to inspect the water quality.

In some examples, the carrier 40 may be designed with dimensions not exceeding 50 cm in length, width, and height. In this case, it may be easily carried by the user. For example, the boat-shaped floating device 1 may have a length designed to not exceed 30 cm, a width not to exceed 15 cm, and a height (the sum of the support portion 201 and the carrier 40) not to exceed 40 cm.

As shown in FIG. 4 and FIG. 5, in some examples, the floating device 1 may further include a driving module 50.

As shown in FIG. 4, in some examples, the driving module 50 is mounted on the carrier 40 to drive the floating device 1 to move in the water. In this case, the floating device 1 may be moved to different positions in the water areas for inspection by the driving module 50, and the floating device 1 may also be moved to a position that is easy for retrieving the floating device 1 to facilitate its recovery.

In some examples, the driving module 50 may include a motor (electrical machinery) and a propeller.

In some examples, the driving module 50 may drive the floating device 1 to move in the water by the means of at least one of airflow or water flow. For example, when the airflow method is adopted, the aforementioned propeller may be designed on the carrier 40 of the floating device 1 without touching the water surface, the airflow formed as the propeller is activated may drive the floating device 1 to move in the water. For another example, when the water flow method is adopted, the aforementioned propeller may be designed under the carrier 40 of the floating device 1 and immersed in the water, the water flow formed when the propeller is activated may drive the floating device 1 to move in the water.

In some examples, the driving module 50 may be controlled by the control module 303. In other examples, the driving module 50 may be controlled by the remote control 70. In this case, the driving module 50 may be remotely controlled to drive the floating device 1 to move in the water, thereby facilitating the user to adjust and control the floating device 1 to perform water quality inspection in different water areas.

As shown in FIG. 4 and FIG. 5, in some examples, the floating device 1 may further include a pulley module 60.

As shown in FIG. 4, in some examples, the pulley module 60 is mounted on the carrier 40 of the floating device 1, which is used to wrap around the second end 1012 of the first electrode 101 and the second end 1022 of the second electrode 102 of the aforementioned inspection module 10, so that the first end 1011 of the first electrode 101 and the second end 1021 of the second electrode 102 are immersed in the water at different depths. In some examples, the first end 1011 of the first electrode 101 and the first end 1021 of the second electrode 102 of the inspection module 10 may be placed in the water at different depths by the pulley module 60, thereby facilitating the user to inspect the water bodies of different depths.

In some examples, the pulley module 60 may be controlled by the control module 303. In other examples, the pulley module 60 may be controlled by the remote control 70. In this case, by remotely controlling the pulley module 60, the first end 1011 of the first electrode 101 and the first end 1021 of the second electrode 102 of the inspection module 10 may be placed in the water at different depths, thereby facilitating the user to adjust and control the floating device 1 to perform water quality inspection in the water at different depths.

As shown in FIG. 3 and FIG. 5, in some examples, the floating device 1 may further include a remote control 70. In some examples, the remote control 70 may be used to remotely control the driving module 50 to drive the portable floating device 1 to move in the water. In some examples, the remote control 70 may be used to remotely control the pulley module 60 to wrap the second end 1012 of the first electrode 101 and the second end 1022 of the second electrode 102 so that the first end 1011 of the first electrode 101 and the first end 1021 of the second electrode 102 are immersed in the water at different depths. In this case, by controlling the driving supply module 50 and the pulley module 60 via the remote control 70, thereby it is facilitated for the user to adjust and control the position of the floating device 1 in the water and the depth at which the inspection module 10 of the floating device 1 is immersed in the water.

In some examples, the remote control 70 may be a common remote control device or an electronic device with control functions such as the user's mobile phone, tablet computer, etc.

In some examples, the remote control 70 may also control the operation of the microcontroller 30. For example, when the remote control 70 is used by the user to control the floating device 1 to reach a designated area in the water and to control the two electrodes of the inspection module 10 to a specific depth, microcontroller 30 may further be controlled by the remote control 70 to start or pause the water quality inspection task of the floating device 1.

According to the present disclosure, a water quality inspection method and a floating device 1 for water quality inspection based on a microcontroller 30 may be provided. The floating device 1 is simple in structure and thereby suitable for the needs of rapid inspection of water qualities during people's outdoor activities, at the same time, it's capable of adaptively adjusting the voltage parameters according to the usage scenario of the electrode to reduce the formation of reactants so as to improve the inspection accuracy.

Although the present disclosure is specifically described with reference to the accompanying drawings and examples, but it may be understood that the above description does not restrict this disclosure in any way. A person skilled in the art may make deformations and changes to the present disclosure as needed without deviating from the essence and scope of the present disclosure, and such deviations and changes fall within the scope of the present disclosure.

Various embodiments of the disclosure may have one or more of the following effects. In some embodiments, the effect of digital quantity control may be achieved by obtaining a predetermined volume of fluid for injection so that the fluid may be delivered with high precision. In other embodiments, the disclosure may be able to provide a medical device and a medical system for conveying fluids with high precision. The medical device controls the conveyance of the fluid by a digital quantity (i.e., a predetermined volume of fluid) so that it may be featured with high precision of fluid conveyance compared to the prior art. In further embodiments, the floating device may be simple in structure, thereby being suitable for the needs of rapid water quality inspection during people's outdoor activities. At the same time, it may be capable of adaptively adjusting the voltage parameters according to the usage scenario of the electrodes to reduce the formation of reactants, thereby improving the inspection accuracy. In some embodiments, the present disclosure may provide a portable floating device for water quality inspection based on a microcontroller, the floating device may be simple in structure, thereby being suitable for the needs of rapid water quality inspection during people's outdoor activities. At the same time, it may be capable of adaptively adjusting the voltage parameters according to the usage scenario of the electrode to reduce the formation of reactants, thereby improving the inspection accuracy.

Many different arrangements of the various components depicted, as well as components not shown, are possible without departing from the spirit and scope of the disclosure. Embodiments of the disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

1.-18. (canceled)

19. A water quality inspection method for inspecting water quality using a floating device of water quality inspection based on a microcontroller, wherein:
the floating device comprises a first electrode and a second electrode;
an electrolyte content in the water is analyzed by the floating device via applying an alternating excitation voltage between the first electrode and the second electrode and based on an intensity of a current formed between the first electrode and the second electrode; and
the inspection method comprises:
within a preset time interval, acquiring a first inspection value using the floating device for inspection, the first inspection value comprising an electrolyte content acquired from the water by the floating device at a first moment;
charactering a water quality condition at the first moment based on the first inspection value;
adjusting a frequency and an amplitude of the excitation voltage based on the first inspection value;
acquiring a second inspection value by inspecting based on the adjusted excitation voltage, the second inspection value comprising an electrolyte content acquired from the water by the floating device at a second moment; and
characterizing the water quality condition at the second moment based on the second inspection value.

20. The water quality inspection method according to claim 19, wherein adjusting the frequency and the amplitude of the excitation voltage is based on an inspection module and the first inspection value.

21. The water quality inspection method according to claim 20, wherein acquiring a frequency and an amplitude of the excitation voltage at a slowest formation of metal reactants on an electrode surface at a plurality of unit times after the first electrode and the second electrode are energized under a plurality of different electrolyte contents through machine learning, and outputting the inspection module.

22. The water quality inspection method according to claim 19, wherein:
the first moment and the second moment are within the preset time interval; and
an interval between the first moment and the second moment does not exceed the preset time interval.

23. The water quality inspection method according to claim 22, wherein:
there are a plurality of preset time intervals;
the plurality of preset time intervals are different from each other;
a preset time interval is selected based on the first inspection value; and
a plurality of the first inspection values and the second inspection values are obtained from the plurality of preset time intervals via Sliding Window Method.

24. The water quality inspection method according to claim 19, wherein:
the first inspection value is an electrolyte content acquired by the floating device from a first water position at the first moment, characterizing the water quality condition at the first water position based on the first inspection value; and
the second inspection value is an electrolyte content acquired by the floating device from a second water position at the second moment, characterizing the water quality condition at the second water position based on the second inspection value.

25. The water quality inspection method according to claim 24, wherein:
the first inspection value further comprises a temperature value acquired by the floating device from the water at the first moment; and
the second inspection value further comprises a temperature value acquired by the floating device from the water at the second moment.

26. A floating device for water quality inspection based on a microcontroller, wherein:
the microcontroller has an executable water quality inspection program, when the floating device executes the water quality inspection program, the water quality inspection is conducted using the water quality inspection method according to claim 19;
the floating device comprises an inspection module, an indication module, an analysis module, a power supply module, a control module, and a floatable carrier;
the inspection module, the indication module, the analysis module, the power supply module, and the control module are installed on the carrier;
the inspection module, the indication module, the analysis module, and the control module are electrically connected to each other and powered by the power supply module;
the inspection module has a first electrode and a second electrode arranged opposite to each other, the inspection module is configured such that: during water quality inspection, a first end of the first electrode and a first end of the second electrode are immersed in water, while a second end of the first electrode and a second end of the second electrode are extended and connected to the power supply module; and
the power supply module, the analysis module, and the control module are integrated into the microcontroller, and, when the microcontroller executes the water quality inspection program they are operable such that: the power supply module applies an excitation voltage between the second end of the first electrode and the second end of the second electrode, the analysis module analyzes the electrolyte content in the water based on an intensity of the current formed between the first electrode and the second electrode, and the control module determines the water quality condition based on the electrode content and controls the indication module to emit an intensity indication based on the condition of the water quality.

27. The floating device according to claim 26, wherein, when the microcontroller executes the water quality inspection program, the control module divides the water quality into a plurality of grades by setting preset values.

28. The floating device according to claim 27, wherein:
the indication module comprises a plurality of indicators corresponding one-to-one with the grades; and
when the water quality is inspected, the control module controls the indicators corresponding to the grades to emit intensity indications based on the grades.

29. The floating device according to claim 27, wherein:
the indication module comprises a rod-shaped support portion;
one end of the support portion is installed on a part of the carrier that floats on a water surface; and
the indicator is mounted on the support portion.

30. The floating device according to claim 27, wherein:
the microcontroller has a display and buttons;
the display is used to display the preset values; and
the buttons are used to enter the preset values.

31. The floating device according to claim 26, wherein:
the first electrode and the second electrode are in a strip shape;
the first end of the first electrode is parallel to the first end of the second electrode;
the first electrode or the second electrode is made of a conductive material either in metal or graphite; and
the second end of the first electrode and the second end of the second electrode are wires.

32. The floating device according to claim 26, wherein:
the carrier is made of a solid material with a density less than that of water;
a part of the carrier that floats on a water surface has a chamber; and
the chamber is used to accommodate and secure the analysis module, the power supply module, and the control module.

33. The floating device according to claim 26, further comprising a power module installed on the carrier, the power module being used to drive the floating device to move in the water.

34. The floating device according to claim 26, further comprising a pulley module installed on the carrier, the pulley module being used to wrap the second end of the first electrode and the second end of the second electrode so that the first end of the first electrode and the second end of the second electrode are immersed in the water at different depths.

35. The floating device according to claim 26, further comprising a remote control, the remote control being used to remotely control the power module to drive the floating device to move in the water.

36. The floating device according to claim 26, further comprising a remote control, the remote control being used to remotely control a pulley module to wrap the second end of the first electrode and the second end of the second electrode so that the first end of the first electrode and the second end of the second electrode are immersed in the water at different depths.
